(19) United States
(12) Patent Application Publication
(10) Pub. No.: US 2023/0416723 A1
(43) Pub. Date: Dec. 28, 2023

(54) METHODS OF OLIGONUCLEOTIDE-BASED AFFINITY CHROMATOGRAPHY

(71) Applicant: DONALDSON COMPANY, INC., Bloomington, MN (US)

(72) Inventors: Jinxiang ZHOU, Greenville, SC (US); Graham TEMPLES, Greenville, SC (US)

(21) Appl. No.: 18/036,732

(22) PCT Filed: Nov. 15, 2021
(86) PCT No.: PCT/US2021/059363

§ 371 Date: May 12, 2023

Related U.S. Application Data
(60) Provisional application No. 63/113,594, filed on Nov. 13, 2020.

(51) Int. Cl. C12N 15/10; C07H 1/06

(52) U.S. Cl. CPC C12N 15/101; C07H 1/06

(57) ABSTRACT

Disclosed are methods for purifying polynucleotides from a feed solution using oligonucleotide affinity columns at flowrates between 0.5 CV/min to 1000 CV/min. The methods can include several steps including loading a mixture containing the target polynucleotide onto a chromatography media, e.g., a column, that carries a macroporous support with an oligonucleotide affinity ligand bonded to a surface. The affinity ligand can hybridize the targeted polynucleotide and allow for separation and purification of the target.

### CROSS REFERENCE TO RELATED APPLICATION

This application claims filing benefit of U.S. Provisional Patent Application Ser. No. 63/113,594, having a filing date of Nov. 13, 2020, entitled “Use of Oligo-DT Based Affinity Membrane,” which is incorporated herein by reference in its entirety.

### BACKGROUND

Therapeutic mRNAs have the potential to advance protein replacement therapies, address a wide variety of pathologies, increase vaccine safety, and shorten vaccine timelines, which is particularly important in pandemic scenarios. For example, Moderna® developed the first vaccine for clinical trials in response to Coronavirus Disease-19 (COVID-19) in the US using their vaccine messenger ribonucleic acids (mRNA) platform in record time, taking only three months to transition from discovery to clinical trials. However, according to the CEO of CureVac, a company pioneering mRNA-based medicines, the quantity and quality, i.e., consistent purity, of mRNA remain a bottleneck for their production. Particularly, the lack of high throughput downstream purification processes is a major challenge in the upscaling of industrial mRNA production.

An example of existing purification processes is chromatography processing used in the purification of mRNA from cellular extracts, in vitro transcription (IVT) reactions, or produced from other bioengineered or synthetic means. For adequate purification, the mRNA must be separated from proteins, nucleic acids, and/or other components from the cell or media as well as additives or solvents used in extraction from the host cell. In purifications from IVT reactions or other synthetic means, capping components, free nucleotides, enzymes such as T7 polymerase, RNase inhibitors (if used), template DNA, as well as any non-mRNA components and/or non-polyadenylated RNA must be removed during purification.

Resin chromatography columns that include a solid phase in the form of individual particles (or resins) have been used in scalable antibody production. However, therapeutic mRNAs (300-1,000 kDa) are much larger than antibodies (˜150 kDa). For instance, as illustrated in FIG. 1, therapeutic mRNA usually possesses a 5-300 nucleotide poly-adenylic acid (poly-A) tail, an essential element to the protein translation process in conjunction with untranslated regions (UTR), caps, and the coding region. Due to such large sizes, the effective surface area and capacity of resin columns are much lower for mRNA than that for antibody purification.

Oligo-deoxythymidine (oligo-dT) ligands have been recognized as an effective affinity ligand to isolate polyadenylated mRNA from feed streams via hybridization following Watson-Crick base-pairing between adenine in the poly-A tail and deoxythymidine in oligo-dT, as shown in FIG. 2. Oligo-dT affinity-based resin chromatography products have been suggested, but unfortunately, they are characterized by low-to-moderate binding capacity of 0.6 to 5 mg mRNA/mL resin for mRNA in the range of 200-4,000 bases in length. Moreover, these resin products require long residence time (on the order of minutes) to perform effectively due to slow mass transfer into the small pores of the solid phase and poor flow properties of the resin-based columns. Additionally, resin-based columns are limited by the maximum operable flow rates, generally under 1 column volume (CV)/min. Bed compression and/or structural deformation of the resin also results in suboptimal performance as well as increased back pressures. Together, the low-to-moderate binding capacity and long residence times of resin columns result in unsatisfactory purification productivity.

Anion-exchange chromatography products have also been proposed for mRNA purification. While anion-exchange chromatography products can provide higher binding capacity as compared to affinity-based systems, it is extremely difficult to elute mRNA from the column with high yield, making them an unappealing alternative.

Advective separation media, such as monoliths and macroporous membranes, have been shown to provide at least ten times faster processing speed than resins for the purification of many biologics. For example, BIA Separations' monolith-based oligo-dT affinity column product moderately reduces residence time (recommended residence times between 12 and 48 seconds, minimum residence of 3.3 seconds).

While the above describes improvement in the art, room for further improvement exists. What is needed in the art are affinity-based membrane chromatography products for mRNA purification. For instance, an oligo-dT based affinity membrane chromatography product for mRNA purification would be of great benefit in the art.

### SUMMARY

According to one embodiment, disclosed is a method for purifying a polynucleotide, e.g., an mRNA, a DNA, etc. A method can include loading a feed solution comprising the polynucleotide onto a chromatographic media. The chromatographic media can include a macroporous support that, in turn, can include an oligonucleotide affinity ligand on a surface of the macroporous support. The oligonucleotide affinity ligand can include a nucleotide sequence that is complementary to a nucleotide sequence of the polynucleotide. As such, as the feed solution flows through the media, the targeted polynucleotide can be retained via hybridization with the affinity ligand and impurities of the feed solution can pass through the chromatographic media. The chromatographic media can exhibit a dynamic binding capacity of from about 0.2 mg polynucleotide/mL to about 15 mg polynucleotide/mL and the methods can be carried out at a high flow rate, e.g., from about 0.5 column volumes (CV)/minute to about 1000 CV/min. A method can also include collecting the polynucleotide following separation of the polynucleotide from the macroporous support, e.g., following elution of the polynucleotide from the macroporous support.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 schematically illustrates the structure of a typical mRNA coding a human protein including the cap, 5′ untranslated region (UTR), coding region, 3′ UTR, and poly-A 3′ tail.

FIG. 2 shows the hydrogen bonding structure between adenine and thymine bases upon hybridization.

FIG. 3 shows mRNA dynamic binding capacity of oligo-dT affinity membranes disclosed herein with different pore sizes at 5 CV/min.

FIG. 4 shows mRNA dynamic binding capacity of oligo-dT affinity membranes disclosed herein at different flow rates, up to 500 CV/min.

FIG. 5 shows purification yields for a 0.1 mL device at various flow rates through 80 CV/min.

FIG. 6 shows theoretical and relative loading step productivity (mg bound up to DBC_{10%}/residence time in minutes) of various oligo-dT products.

FIG. 7 compares the 10% dynamic binding capacity value (DBC_{10%}) of a commercially available resin-based separation media at 2 different flow rates for 2 different mRNA targets.

FIG. 8 provides the DBC_{10%} value obtained in a method as described herein for mRNA targets of different sizes at multiple different flow rates.

FIG. 9 illustrates results of a 20-cycle bind-and-elute study including clean in place protocols to determine long-term binding capacity of disclosed methodologies.

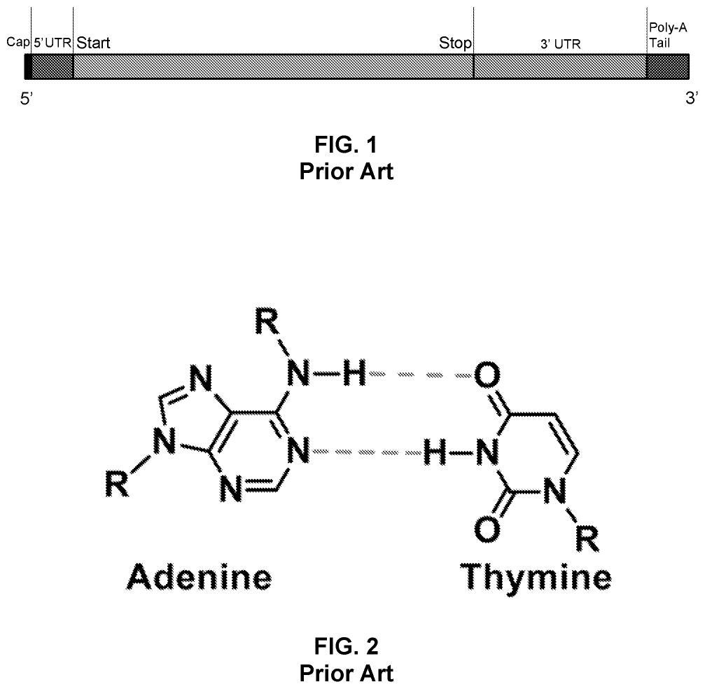

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

In general, disclosed herein are affinity-based chromatographic processes utilizing a separation device that includes an oligonucleotide ligand on a macroporous support. Disclosed methods can provide high binding capacity in performance of polynucleotide purifications including high impurity clearance with low backpressure at flowrates of from about 0.5 CV/min to about 1000 CV/min. Disclosed methods can beneficially provide for full target sequence recovery values at about 80% or higher.

Disclosed methods can be utilized for purifying polynucleotides rapidly and efficiently using oligonucleotide-based affinity media. By way of example, disclosed methods can successfully purify target polynucleotides at flowrates from about 0.5 CV/min to about 1000 CV/min. For one embodiment, a method can be carried out at a flowrate of from about 0.5 CV/min to about 500 CV/min. For one embodiment, a method can be carried out at a flowrate of from about 1 CV/min to about 1000 CV/min. For one embodiment, a method can be carried out at a flowrate of from about 1 CV/min to about 500 CV/min. For one embodiment, a method can be carried out at a flowrate of from about 5 CV/min to about 1000 CV/min. For one embodiment, a method can be carried out at a flowrate of from about 5 CV/min to about 500 CV/min.

Devices for use in disclosed methods can include macroporous membrane support materials that include an oligo-nucleotide affinity ligand thereon. In one embodiment, disclosed methods can utilize macroporous oligonucleotide-based affinity media as described in U.S. Patent Application Publication No. 2020/0188859 to Zhou et al., which is incorporated herein by reference in its entirety. By way of example, a macroporous support can include, without limitation, polyolefins membranes, polyether sulfone membranes, poly(tetrafluoroethylene) membranes, nylon membranes, fiberglass membranes, hydrogel membranes, hydrogel monoliths, polyvinyl alcohol membranes; natural polymer membranes, cellulose membranes (e.g., cellulose ester membranes, cellulose acetate membranes, regenerated cellulose membranes, cellulosic nanofiber membranes, cellulosic monoliths, membranes containing substantially (e.g., about 90 wt. % or greater) cellulose or its derivatives), filter paper membranes, and combinations thereof.

A macroporous support can be derivatized to exhibit an oligo-nucleotide affinity ligand at a surface, optionally via a naturally occurring reactive site or a coupling group including a reactive site that has been bonded to the membrane. For instance, a macroporous support can be subjected to a multi-step derivatization process in which a membrane is soaked in a swelling solvent (e.g., dimethyl sulfoxide, acetonitrile, tetrahydrofuran, dimethylformamide, etc.) in conjunction with an activating agent (e.g., N,N′-disuccinimidyl carbonate) to add a reactive site to the membrane. Following, the reactive site can be reacted directly with an affinity ligand or optionally reacted with an intermediate group (e.g., an intermediate group comprising further reactivity toward an affinity ligand optionally in conjunction with an alkyl chain as a spacer group, discussed further herein) to form the macroporous support for use as described herein. Of course, other formation methods as are generally known in the art may alternatively be utilized to provide a macroporous support for use in disclosed methods. In general, a macroporous support for use in disclosed methods can include a specific surface area of from about 1 m²/mL to about 20 m²/mL.

In general, a method can utilize an oligo-nucleotide affinity ligand that includes a sequence (e.g., about two or more individual sequences of the entire ligand) that is a complementary sequence to a target polynucleotide. As will be known in the art, the complementary portion of an affinity ligand and a target need not extend the entire length of the two, and a portion of each can hybridize, optionally with discontinuous segments of each hybridizing with one another. For instance, in those embodiments in which an affinity ligand includes a modified base (e.g., a PNA or LNA base as discussed further herein), that particular base may not hybridize with a base of the target, but a sequence of bases on one or both sides of the modified base can hybridize with bases of the target polynucleotide. For instance, a macroporous support can carry oligonucleotide ligand moieties that can bind targeted polynucleotides with a dynamic binding capacity of from about 0.2 mg polynucleotide (e.g., RNA)/mL to about 15 mg polynucleotide/mL in some embodiments.

In one embodiment, a method can utilize an oligo-dT of from about 5 to about 100 bases in length as an affinity ligand, however, longer lengths can be used in some embodiments. For instance, an oligonucleotide affinity ligand immobilized to the affinity media can be from about 5 to about 25 bases in length in some embodiments, such as from about 5 to about 20 bases in length, such as from about to about 100 bases in length, such as from about 10 to about 50 bases in length, such as from about 10 to about 40 bases in length, such as from about 10 to about bases in length, such as from about 10 to about 20 bases in length, such as from about 20 to about 100 bases in length, such as from about 20 to about 40 bases in length, such as from about 20 to about 30 bases in length.

In some embodiments, a spacer can be covalently bound between an macroporous support and an oligonucleotide affinity ligand. In general, a spacer can include a carbon-based monomer or oligomer. By way of example, a spacer can include a carbon-based monomer (e.g., —CH₂—) or can be a carbon-based oligomer including a chain length of up to about 50 carbon atoms. For instance, a spacer can include a chain length of up to about 20 carbons or up to about 10 carbons in length in some embodiments. For one embodiment, a carbon-based spacer can be from about 5 to about 50 carbons in length, such as from about 5 to about 20 carbons in length, such as from about 5 to about 10 carbons in length.

In one embodiment, an affinity ligand can include one or more base substitutions as compared to natural bases of a complementary sequence to the target polynucleotide that can alter the performance of the ligand. One such modification is use of Locked Nucleic Acid (LNA) bases. LNAs are modified RNA bases with a covalent bond linking the 2′ oxygen and 4′ carbon on the ribose sugar.

In one embodiment, a Peptide Nucleic Acid (PNA) ligand can be utilized as an affinity ligand. PNAs utilize peptide bonds to connect bases without a negatively charged backbone. In addition to this configuration enhancing affinity, the lack of negative charge on the ligand can allow for binding operations and purification processes to be performed with feeds exhibiting little conductivity.

Base modification can be used as a substitution for one or more nucleotides of an affinity ligand. Such modification can further enhance interactions between the affinity ligand and the target nucleotide and allow for high flow rates to be used to improve chromatographic productivity. In one embodiment, an affinity ligand can include a single modified base. In other embodiments, an affinity ligand can include multiple modified bases. For instance, an affinity ligand can include an LNA or a PNA base at every other position, every third position, every fourth position, or every fifth position of an affinity ligand. Additionally, there can be discontinuous tracts of modified bases interspersed with natural bases, for example, a tract of 3 LNA or PNA alternating with tracts of 3 natural bases; however, tracts need not be equally proportioned or regularly spaced, for example, a tract of 3 LNA or PNA alternating with a tract of 5 natural bases or repeats of a tract of 3 LNA or PNA followed by 5 natural bases, followed by 2 LNA or PNA, followed by 7 natural bases. Moreover, base substitutions can be of the same type or of different types. For instance, an affinity ligand can include multiple base substitutions, with every base substitution being an LNA base or a PNA base or the multiple base substitutions can include a mixture of both LNA bases and PNA bases in any combination, though in other embodiments, only one type of substitution may be included in an affinity ligand that has been modified from the complementary sequence of the target molecule of traditional, non-modified bases, e.g., only one or more LNA substitution, only one or more PNA substitution, or only one substitution of a natural base for a modified base, examples of which are provided further below.

In some embodiments, base modification can allow for binding at reduced conductivity potentially, which can reduce the need for an extensive post-binding wash step and can further increase processing speed of a purification protocol. For instance, an oligonucleotide ligand including full PNA substitutions can maintain hybridization properties at conductivities below 1.5 mS/cm. Base modifications can promote greater complementary base recognition in some embodiments, which can provide opportunities for novel nucleic acid separation techniques such as separation of single base replacement mutants or targeted purifications of sequences without poly-adenylation. Of course, full PNA or LNA base substitution of an oligonucleotide affinity ligand is not required, and base substitutions can be for no, one, or multiple bases of an oligonucleotide affinity ligand. In one embodiment, an affinity ligand, including partial or full LNA or PNA substitutions, for individual base of an oligonucleotide ligand can increase the potential for targeted purifications of double stranded nucleic acids resulting from triplex formation with the oligonucleotide ligand and the target via Hoogsteen hydrogen bonding interactions.

Modifications encompassed herein are not limited to PNA and/or LNA base substitutions. For instance, base modifications can include base substitutions for cytidine or uridine bases of an oligonucleotide affinity ligand including, without limitation, one or more of 5-Iodocytidine-5′-triphosphate, 5-methylcytidine-5′-triphosphate, 2-thiocytidine-5′-triphosphate, 6-azacytidine-5′-triphosphate, 5-bromocytidine-5′-triphosphate, 5-aminoallylcytidine-5′-triphosphate, pseudoisocytidine-5′-triphosphate, N⁴-methylcytidine-5′-triphosphate, 5-carboxycytidine-5′-triphosphate, 5-formylcytidine-5′-triphosphate, 5-hydroxymethylcytidine-5′-triphosphate, 5-hydroxycytidine-5′-triphosphate, 5-methoxycytidine-5′-triphosphate, thienocytidine-5′-triphosphate, 5-bromo-2′-deoxycytidine-5′-triphosphate, 5-propynyl-2′deoxycytidine-5′-triphosphate, 5-iodo-2′-deoxycytidine-5′-triphosphate, 5-methyl-2′-deoxycytidine-5′-triphosphate, 2′-deoxy-P-nucleoside-5′-triphosphate, 5-hydroxy-2′deoxycytidine-5′-triphosphate, 2-thio-2′-deoxycytidine-5′-triphosphate, 5-aminoallyl-2′-deoxycytidine-5′-triphosphate psudouridine-5′-triphosphate, 2′-O-methylpsudouridine-5-triphosphate, N¹-methylpseudouridine-5′-triphosphate, N¹-ethylpseudouridine-5′-triphosphate, N¹-methyl-2′-O-methylpseudouridine-S-Triphosphate, N¹-methoxymethylpseudouridine-5′-triphosphate, or N¹-propylpseudouridine-5′-triphosphate.

Oligonucleotide ligand-based affinity membrane columns utilized in disclosed methodologies can operate either in a bind-and-elute mode or in a flow-through mode.

Process productivity of a separation can be defined using the below equation. In the equation, Vₜₒₜ represents the total volume of solution passing through the column during a separation protocol, including load, rinse, elution, and regeneration steps. BV represents the oligonucleotide medium bed volume. Loading volume can be proportional to dynamic binding capacity of the oligonucleotide medium. Thus, process productivity can increase with increasing binding capacity and decreasing residence time.

$$Productivity = \frac{Polynucleotide captured}{Cost of time} = \frac{Loading volume \times polynucleotide concentration \times yield}{( \frac{{V}_{tot}}{BV} ) \times residence time}$$

Disclosed methods can provide higher productivity by a factor of 10 or greater as compared to existing resin chromatography-based methods. For instance, as shown in FIG. 6, methods as disclosed herein (designated by use of Membrane 1, 2 and 3 of FIG. 6) can provide higher binding capacity at much lower residence time and greatly improved productivity as compared to methods utilizing previously known resin-based separation materials. Further, oligonucleotide ligand-based affinity chromatography for polynucleotide purification as disclosed herein may be operated at flowrates from 0.5 CV/min to 1000 CV/min in some embodiments; whereas the current oligo-dT resin column products operate at flowrates below 1 CV/min. For instance, current oligo-dT resin column products can require a residence time of 360 seconds, whereas disclosed methods can be carried out using a residence time of much faster, as indicated in FIG. 6. In some embodiments, oligonucleotide ligand-based affinity chromatography for polynucleotide purification as described herein can operate effectively at a residence times as low as 0.06 seconds.

The size (i.e., internal volume) of a separation device for disclosed methods is not particularly limited. In general, a preferred device size can be selected based upon the scale of the preparation, with difference device sizes used for different scale preparations. As such, the volume of the macroporous support of a separation protocol can also vary. In one embodiment, the volume of a macroporous support can be from about 0.025 mL to about 100 liters, such as from about 0.2 mL to about 5 mL, such as from about 1 mL to about 100 mL, such as from about 100 mL to about 1 liter, such as from about 0.2 mL to about 1 liter, such as from about 0.2 mL to about 10 liters, such as from about 1 liter to about 10 liters, such as from about 10 liters to about 100 liters.

An oligonucleotide affinity-based purification process as disclosed herein can generally include multiple steps. One step of a protocol can include loading a feed solution containing polynucleotides for separation onto a chromatographic media that can include a macroporous support. A feed solution fed to the chromatographic media can in some embodiments exhibit a conductivity. For instance, in one embodiment, such as when an affinity ligand incorporates one or more base substitutions (e.g., LNA and/or PNA substitutions), the conductivity of the feed solution can be up to about 3.35 mS/cm, or even higher in some embodiments. In one embodiment, a conductivity of a feed can be from 0 to 3.35 mS/cm, such as from about 1.5 to about 3.35 mS/cm. As stated previously, at least one substitution of the oligonucleotide affinity ligand to a LNA base and/or a PNA base can be utilized to improve aspects of a separation protocol when considering a feed solution exhibiting a conductivity.

A targeted polynucleotide of a feed solution can have any structure or base content. In one embodiment, a purification target can be single stranded RNA or DNA. This is not a requirement, however, and in one embodiment, the purification target can be double stranded DNA, double stranded RNA, hybridized DNA/RNA duplexes, DNA/peptide conjugates, RNA/peptide conjugates, DNA/polypeptide conjugates, or RNA/polypeptide conjugates. In one embodiment, a targeted polynucleotide can have a size of from about 300 to about 5,000 bases. For instance, a targeted polynucleotide can be a single stranded or double stranded RNA or DNA of about 800 bases/base pairs (if double stranded) in length or greater, such as from about 500 bases/base pairs to about 15,000 bases/base pairs in some embodiments, such as from about 1,000 bases/base pairs to about 12,000 bases/base pairs, such as from about 4,000 bases/base pairs to about 10,000 bases/base pairs, such as from about 800 bases/base pairs to about 4,000 bases/base pairs, such as from about 10,000 bases/base pairs to about 15,000 bases/base pairs in some embodiments, though longer and shorter target polynucleotides are encompassed herein.

Other characteristics of a feed solution are not particularly limited. In one embodiment, the pH of the feed solution can range from about 1 to about 8.5. In one embodiment, the pH of the feed solution can range from about 6 about 10. In some embodiments, a high pH feed solution can be utilized in a protocol targeting a DNA, and in some embodiments, a lower pH feed solution can be utilized in a protocol targeting an RNA, though this is not a requirement of disclosed methodologies.

In some embodiments, a separation protocol can include a wash step following a binding step and prior to elution. For instance, a wash step can be utilized to clear one or more impurities from the media prior to elution. In one embodiment, a wash step can utilize a washing fluid exhibiting a conductivity (e.g., by the addition of one or more suitable salts as are known in the art). In such an embodiment, a conductive wash (e.g., high salt content) can be utilized in a protocol that includes a low or no conductive feed solution during the binding step or alternatively with a relatively high conductive feed solution, i.e., high conductive binding. For instance, the conductivity of a fluid used in a wash step can range in some embodiments from a non-conductive wash solution to about 3.35 mS/cm. For instance, a fluid used for a wash step can exhibit a conductivity that is essentially the same (e.g., within about 10% or less) of a conductive or non-conductive feed stream, or that differs from that of a feed stream, for instance, a wash fluid can exhibit a conductivity that differs from the conductivity of the feed stream by about 1.5 mS/cm or less, or by about 3.35 mS/cm, such as, from about 1.5 to about 3.35 mS/cm in some embodiments. Of course, a wash step is not a requirement of disclosed protocols, and in one embodiment, a wash step under non-loading buffer conditions need not be carried out. For instance, in those embodiments in which an oligonucleotide affinity ligand includes one or more PNA base substitutions in the ligand, it may not be necessary or desired to carry out a wash step under non-loading buffer conditions.

A separation protocol can include a step of eluting the targeted polynucleotide from the chromatographic media medium. Eluents as are generally known in the art can be utilized in disclosed methods including, without limitation, water (e.g., deionized water, RNase-free water), tris(hydroxymethyl)aminomethane hydrochloride (Tris-HCl) (e.g., 10 mM Tris-HCl pH 7.0-7.5, generally RNase free), etc.).

Generally, elution of a targeted polynucleotide can be carried out after impurities are separated significantly from the polynucleotide. In one embodiment, an elution solution can exhibit a conductivity. By way of example, an elution solution can exhibit a conductivity of about 1.5 mS/cm or less, such as from 0 to about 1.5 mS/cm. In one embodiment, the temperature of an elution solution can be from ambient (i.e., room temperature or about 25° C.) to about 90° C., such as from about 25° C. to about 65° C., or from about 15° C. to about 90° C. in some embodiments. In one embodiment, an elution solution can have a temperature greater than about 40° C. For instance, a higher temperature elution solution may be utilized in one embodiment in which an oligonucleotide affinity ligand includes one or more PNA bases on the ligand. In general, the pressure across the separation media (e.g., the trans-membrane pressure or trans-column pressure) during an entire separation protocol can be about 1 MPa or less, such as about 0.5 MPa or less in some embodiments.

Disclosed separation protocols can provide high capacity binding at high flow rates for purification of polynucleotides of any size. Moreover, disclosed separation protocols can provide long-term binding capacity, with binding capacity retained at a value of about 80% or greater, about 85% or greater, about 90% or greater, about 95% or greater, 96% or greater, or 97% or greater over multiple bind-and-elute cycles, e.g. over 20 bind-and-elute cycles, over 50 bind-and-elute cycles, or over 100 bind-and-elute cycles, in some embodiments.

The present disclosure may be better understood with reference to the Examples set forth below.

### Example 1

mRNA dynamic binding capacity of oligo-dT based affinity membrane columns was examined. In the example, the 10% dynamic binding capacity value (DBC_{10%}) was determined. The DBC_{10%} represents the mass of target bound per unit volume of chromatography media when the target concentration in the effluent from the membrane bed reaches 10% of the target concentration in the feed solution.

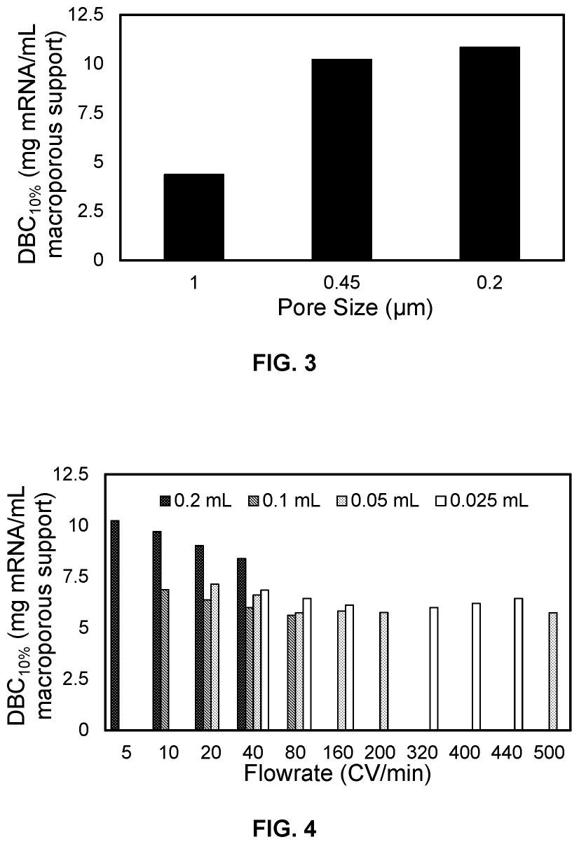

FIG. 3 shows the DBC_{10%} of oligo-dT based membrane columns using membranes having three different pore sizes. The membranes were formed according to methods described in US Patent Application Publication No. 2020/0188859, previously incorporated herein by reference. Briefly, the membranes included a 25-base oligo-dT affinity ligand and included a 6 C spacer between the macroporous matrix and the oligo-dT affinity ligand. The nominal pore sizes of the three membrane columns were 0.2 μm, 0.45 μm, and 1.0 μm. The targeted mRNA was a green fluorescent protein (GFP) mRNA with ˜800 bases and a poly-A tail. The mRNA was dissolved in a 50 mM phosphate, 250 mM NaCl, pH 7.0 buffer feed solution. The mRNA concentration was 0.1 mg/mL in the feed solution. The loading flow rate was fixed at 5 CV/min representing a residence time of 12 seconds. The tests were conducted at room temperature. As indicated in FIG. 3, DBC_{10%} was higher for membrane columns with smaller pore sizes, with DBC_{10%} reaching more than 10 mg/mL as indicated.

### Example 2

Impact of flow rates on mRNA dynamic binding capacity of oligo-dT based affinity membrane columns were examined. Separation media were as described in Example 1, above.

FIG. 4 provides the 10% dynamic binding capacity (DBC_{10%}) of oligo-dT based chromatography media using various flowrates. The feed solution was 0.1 mg/mL of GFP mRNA in 50 mM phosphate 250 mM NaCl, pH 7.0. Residence time is inversely proportional to flow rates for a given chromatography column. In this example, residence time was varied between 12 seconds and 0.12 seconds, which corresponded to flow rates of 5 CVs/min to 500 CVs/min in the relatively small volume columns utilized (FIG. 4). As shown, DBC_{10%} was marginally impacted by the flow rates for a given bed volume.

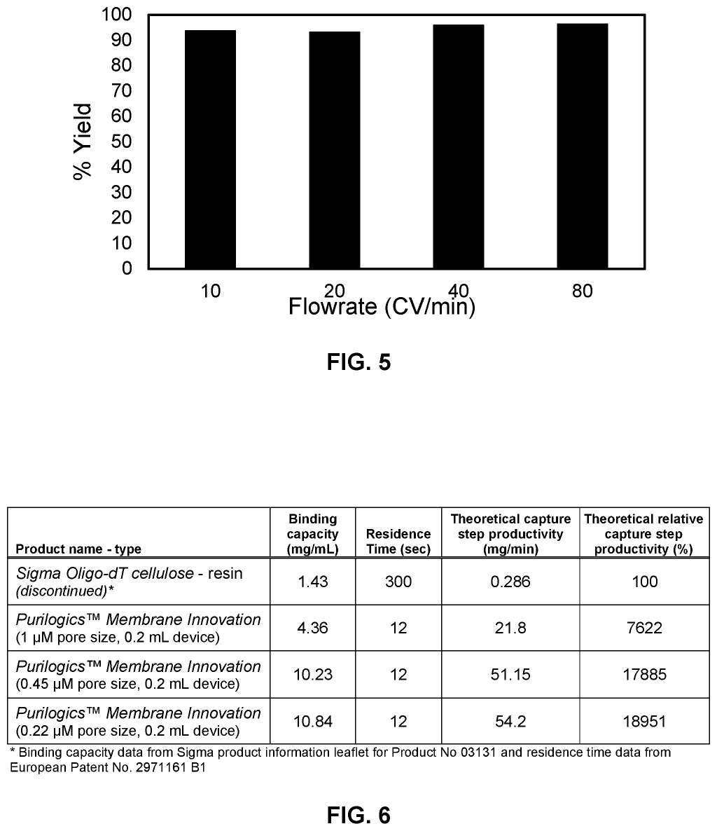

FIG. 5 provides the percent yield (mg mRNA injected/mg mRNA recovered in eluate) for a 0.1 mL device using a 0.45 μm pore size macroporous support with a 25-base oligo-dT with a 6-carbon spacer between the macroporous matrix and the affinity ligand, as prepared as described above. As shown, yields were consistent for flow rates up to at least 80 CV/min.

FIG. 6 includes the theoretical loading step productivity (mg mRNA/minute) for a product as reported in the literature as well as data collected using a 0.2 mL device incorporating a macroporous membrane separation media with either 0.2 μm, 0.45 μm, or 1.0 μm pore size and functionalized with a 25-base oligo-dT with a 6 C spacer as described and operated at 5 CV/min. As indicated, loading step productivities for disclosed methods can exceed performance previously known methods utilizing resin-based separation materials using 1/200^{th} of the operable speed.

### Example 3

Binding capacity of targeted mRNA was examined for different sized targets and for different separation media. Materials examined included a commercially available product (POROS™-OdT Resin) and a macroporous membrane media having a 0.45 μm pore size functionalized with a 25-base oligo-dT affinity ligand attached to the macroporous membrane via a 6 C spacer. Targeted mRNA includes a 4000-base mRNA and an 800-base mRNA. The feed solutions included 0.1 mg/mL of one of the targeted mRNA in 50 mM phosphate 250 mM NaCl, pH 7.0.

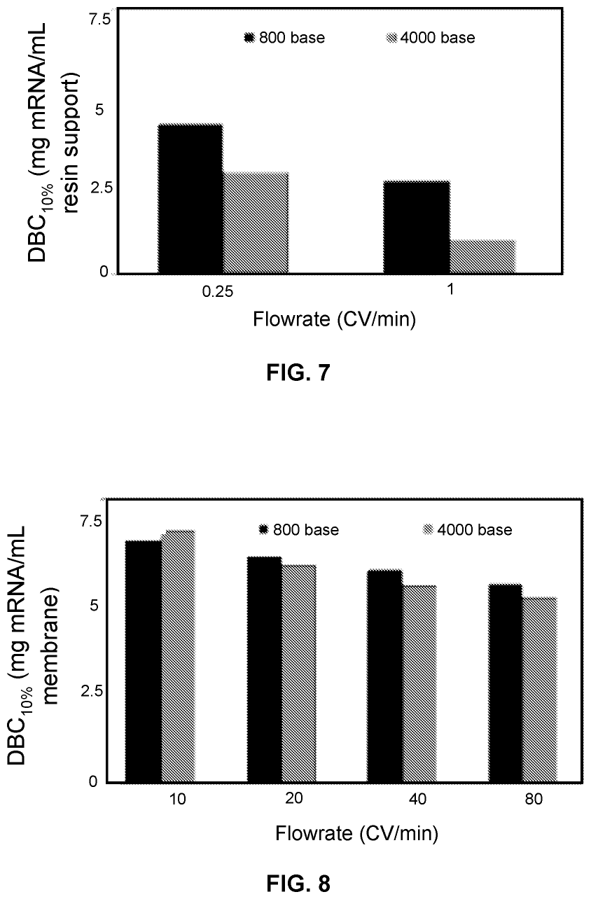

The commercially available product is recommended for use at a flow rate of 0.25 CV/min. Separation protocols were run using this commercially available product for both mRNA targets at the recommended flow rate of 0.25 CV/min, as well as at a higher flow rate of 1 CV/min. Results are shown in FIG. 7. As indicated, the binding capacity for the 4000-base mRNA was 32% lower than that for the 800-base mRNA at the recommended flow rate of 0.25 CV/min. Increasing the flow rate beyond the recommended limit resulted in a 38% capacity reduction for the 800-base mRNA and a 66% capacity reduction for the 4000-base mRNA.

The membrane-based separation material was also examined for separation of the two differently sized mRNA and at multiple flowrates from 10 CV/min to 80 CV/min. Results are shown in FIG. 8. As shown, the binding capacity for the 4,000-base mRNA was only 7% lower than that for the 800-base mRNA. Moreover, there was only a 20% reduction in capacity for the 800-base mRNA protocol and a 26% reduction in capacity for the 4,000-base mRNA protocol from the lowest to the highest flow rate (10-80 CV/min).

The purification protocols were 10 to 160 times faster with the disclosed methodologies as compared to the resin-based separation protocols under manufacturer recommended conditions. Moreover, disclosed methods led to target recoveries between 93% and 96%. The disclosed methodologies exhibit a more robust performance as compared to resin-based methods, particularly when considering purification of large mRNA targets.

### Example 4

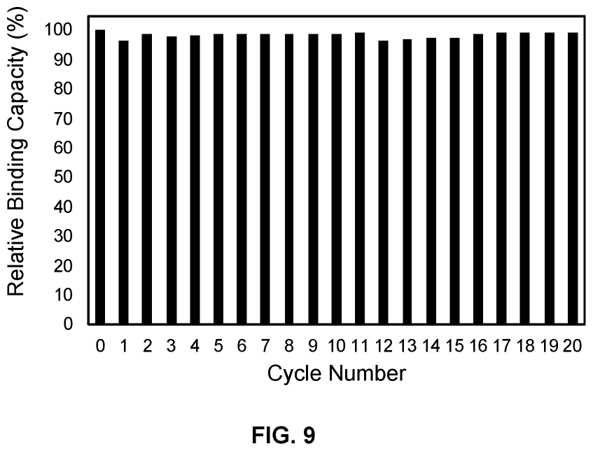

Clean-in-place protocols were carried out on the membrane separation device of Example 4 using 0.1 M NaOH and a contact time of 5 minutes. The protocols were performed for 20 bind-and-elute cycles. FIG. 9 provides the results demonstrating that the binding capacity of the device was maintained at 98% on average of the 20-cycle study, with the lowest value determined to be 96.5% over the course of the study.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art using the teachings disclosed herein.

1. A method for purifying a polynucleotide comprising:
loading a feed solution comprising the polynucleotide onto a chromatographic media, the chromatographic media comprising a macroporous support having a specific surface area of from about 1 m²/mL to about 20 m²/mL, the macroporous support including an oligonucleotide affinity ligand bonded to a surface of the macroporous support and in fluid communication with the feed solution, the oligonucleotide affinity ligand comprising a nucleotide sequence that is complementary to a nucleotide sequence of the polynucleotide, the chromatographic media exhibiting a dynamic binding capacity of from about 0.2 mg polynucleotide/mL to about 15 mg polynucleotide/mL;
flowing the feed solution through the chromatographic media, wherein upon the flowing, the polynucleotide is retained on the macroporous support via hybridization with the affinity ligand and impurities of the feed solution pass through the chromatographic media; and
collecting the polynucleotide following separation of the polynucleotide from the macroporous support.

2. The method according to claim 1, further comprising eluting the polynucleotide off of the macroporous support, wherein the eluent is optionally heated, the method optionally comprising a wash step prior to the eluting.

3. The method according to claim 2, wherein the elution comprises flowing a conductive eluent through the chromatographic media and/or wherein the optional wash step comprises a conductive washing fluid.

4. The method according to claim 1, wherein the feed solution flows through the chromatographic media at a flow rate of from about column volumes per minute (CV/min) to about 1,000 CV/min.

5. The method according to claim 1, wherein the macroporous support comprises a polyolefin, a polyether sulfone, a poly(tetrafluoroethylene), a nylon, a fiberglass, a hydrogel, a polyvinyl alcohol, a natural polymer, a cellulose, a filter paper, or a combination thereof.

6. The method according to claim 1, wherein the macroporous support comprises a membrane.

7. The method according to claim 1, wherein the oligonucleotide affinity ligand is from about 5 to about 100 bases in length.

8. The method according claim 1, wherein the oligonucleotide affinity ligand comprises an oligo-deoxythymidine sequence.

9. The method according to claim 1, wherein the macroporous support comprises a spacer between the oligonucleotide affinity ligand and the surface of the macroporous support.

10. The method according to claim 1, wherein the oligonucleotide affinity ligand comprises one or more base substitutions.

11. The method according to claim 1, wherein the macroporous support defines a volume of from about 0.2 milliliters to about 100 liters.

12. The method according to claim 1, wherein the feed solution is a conductive solution, for instance wherein the feed solution exhibits a conductivity of up to about 3.35 milliSiemens per centimeter (mS/cm).

13. The method according to claim 1, wherein the feed solution exhibits a pH of from about 1 to about 8.5.

14. The method according to claim 1, wherein the method is repeated multiple times, and wherein the binding capacity of the method is retained at a value of about 95% or greater of the initial binding capacity over 20 cycles.

15. The method according claim 1, wherein the polynucleotide comprises a single-stranded RNA, a double-stranded RNA, a single-stranded DNA, a double-stranded DNA, a hybridized DNA/RNA duplex, a DNA/peptide conjugate, an RNA/peptide conjugate, a DNA/polypeptide conjugate, or an RNA/polypeptide conjugates, and further wherein the polynucleotide is from about 500 bases to about 15,000 bases in length.

16. The method according to claim 1, wherein the macroporous support comprises a cellulose selected from a cellulose ester, a cellulose acetate, a regenerated cellulose, a cellulose nanofiber.

17. The method according to claim 1, wherein the oligonucleotide affinity ligand comprises one or more locked nucleic acid bases and/or one or more peptide nucleic acid bases.

18. The method according to claim 1, wherein the feed solution exhibits a pH of from about 6 to about 10.

19. The method according to claim 1, wherein the polynucleotide comprises an mRNA.

20. The method according to claim 1, wherein the polynucleotide is from about 800 bases to about 4,000 bases in length.
